(19) United States
(12) Patent Application Publication
(10) Pub. No.: US 2003/0207339 A1
(43) Pub. Date: Nov. 6, 2003

(54) PCAM-1 marker protein, nucleic acid sequences encoding PCAM-1 and immunoassays for detection of PCAM-1

(75) Inventors: Mark Stearns, Villanova, PA (US); Youji Hu, Gulph Mills, PA (US)

Correspondence Address: MORGAN, LEWIS & BOCKIUS LLP, 1701 MARKET STREET, PHILADELPHIA, PA, 19103-2921, US

(73) Assignee: Philadelphia Health and Education Corporation

(21) Appl. No.: 10/098,992

(22) Filed: Mar. 15, 2002

Related U.S. Application Data
(63) Continuation of application No. PCT/US00/25981, filed on Sep. 21, 2000.
(60) Provisional application No. 60/155,865, filed on Sep. 24, 1999.

(51) Int. Cl. C12Q 1/68; G01N 33/574; G06F 19/00; G01N 33/48; G01N 33/50; C07H 21/04; C12N 9/00; C12P 21/02; C12N 5/06

(52) U.S. Cl. 435/7.23; 435/69.1; 435/183; 435/320.1; 435/325; 536/23.2; 702/19; 530/388.26; 435/6

(57) ABSTRACT

The present invention provides a cancer marker protein, PCAM-1, and polynucleotides which identify and encode this protein. Detection of this marker is useful in diagnosing and prognosticating cancer in a patient. Also provided are expression vectors and host cells for expression of PCAM-1 as well as antibodies raised against the PCAM-1 protein. In addition, a Monte Carlo-like screening assay for identification of specific 8 mer sequences which selectively bind proteins in a crude extrract of tissues, cells or other biological fluids is provided.

### FIELD OF THE INVENTION

The invention relates to nucleic acid probes (i.e., DNA consensus domains), referred to herein as PCAM-1 probe 1 and PCAM-1 probe 2, which specifically bind a newly identified marker protein for cancer, herein referred to as PCAM-1. The invention further relates to the nucleic acid sequence and amino acid sequence of PCAM-1. Also provided are polyclonal and monoclonal antibodies which specifically recognize PCAM-1. The invention further relates to the use of these antibodies in immunoassays designed to detect PCAM-1 protein in the diagnosis, prevention and treatment of diseases relating to disregulated cell growth and proliferation, including cancer.

### BACKGROUND OF THE INVENTION

The existence of chromosomal abnormalities in lymphoid tissue is well established. Chromosomal translocations associated with T cell acute lymphoblastic leukemia (T-ALL) have led to the identification of several potential oncogenes (Rabbitts, T. H. Cell 1991 67:641-644). Many of the T-ALL associated chromosomal translocations have been localized to the T-cell receptor (TCR) genes. Recombination of the immunoglobulin gene takes place at early phase of B-lymphocyte differentiation. The V-(D)-J recombination that joins two or three germline segments (i.e. variable-V; diversity-D; and joining-J) segments into a V-gene exon by site specific recombination contributes to amplification of the V-region diversity. Comparison of the nucleotide sequences of the flanking regions of the V, D, and J segments has shown that two common blocks of nucleotide sequences are conserved (Early et al. Cell 1980 19:981-992) including a heptamer CACTGTG and a T-rich nonamer GGTTTTTGT which are separated by a spacer sequence of either 12 or 23 bases. The homology between the heptamer-spacer-heptamer-nonamer sequences of the T-cell receptor and immunoglobulin genes suggests that the elements, commonly referred to as Break Point Cluster Region or BPCRs, have an important role in V-(D)-J recombination. It is believed that DNA binding protein(s) that would recognize the conserved recombination signal sequence (RS) may be involved in the recombinational machinery that cleaves DNA at the juncture between the signal and coding region sequences and ligates the cleaved ends. Earliest reports disclosed RS proteins as being located in lymphoid cells (Aguilera et al. Cell 1987 51:909-917; Halligan, B. D. and Desiderio, S. V. Proc. Natl Acad. Sci. USA 1987 84:7019-7023; Hamaguchi et al. Nucleic Acid Res. 1989 17:9015-9026; and Mak, C. H. Nucleic Acid Res. 1994 22:383-390). Different RS proteins have been identified more recently. For example, a DNA binding protein for kappaB binding and recognition component of the V(D)J recombination signal sequence has been identified. Activation of this family of transcription factors is thought to provide a mechanism by which oncogenic tyrosine kinases regulate genes with kappaB-controlled gene regulatory elements.

Studies on T cell abnormalities have been particularly informative with respect to recombinase involvement, especially with respect to breakpoints within the chromosome band 11p13. It seems that recombinase is responsible for abnormal chromosomal unions, because often both reciprocal translocated chromosomes have N-region nucleotide addition which is a hallmark of recombinase activity (Alt, F. W. and Baltimore, D. Proc. Natl Acad. Sci. USA 1982 79:4118-4223). These translocations are regarded as mutations of the normal chromosomal joining process.

There are many reports showing a connection between overexpression of genes encoding ribosomal proteins and cancer (Chiao et al. Mol. Carcinog. 1992 5:219-231; Fernandez-Pol et al. J. Biol. Chem. 1993 268:21198-211204; Fernandez-Pol et al. Cell Growth & Differentiation 1994 5:821-825; Fernandez-Pol, J. A. Anticancer Res. 1996 16:2177-2186; Chan et al. Biochem and Biophys. Res. Comm. 1996 228:141-147; Chan et al. Biochem. and Biophys. Res. Comm. 1996 225:952-956; Wool, I. G. Trends in Biochemical Sciences 1996 21:164-165; Wool et al. Biochemistry and Cell Biology 1995 73:933-947; and Vaarala et al. Int. J. Cancer 1998 78:27-32). For example, Chiao et al. (Mol. Carcinog. 1992 5:219-231) found that the S2-ribosomal protein was elevated in head and neck cancer, but barely detectable in normal tissue. Based upon these studies, it is widely believed that ribosomal proteins have a role in elevating protein synthesis in cancer.

Alternatively, it has been proposed that specific leucine zipper sequence motifs or other motifs characteristic of numerous ribosomal proteins may be mutated and that the mutant can then bind to nucleic acids (Fernandez-Pol et al. Anticancer Res. 1996 16:2177-2186; Wool, I. G. Trends in Biochemical Sciences 1996 21:164-165; Wool, I. G. (1997) The ribosomal RNA and Group I introns (R. Green and R. Schroeder, Eds.) R. G. Landes Co., Austin Tex. USA pp.153-178) and either function as a nuclease, control ligation or regulate gene transcription or translation in cancer cells. For example, the rat ribosomal protein S3a is identical to the product of the rat v-fos transformation effector gene (Chan et al. Biochem. and Biophys. Res. Comm. 1996 228:141-147). S3a is involved in initiation of protein synthesis and is also related to proteins involved in the regulation of growth and the cell cycle (Chan et al. Biochem. and Biophys. Res. Comm. 1996 228:141-147). Likewise, the rat ribosomal protein L10 is homologous to a DNA-binding protein to a putative Wilm's tumor suppressor gene (Chan et al. Biochem. and Biophys. Res. Comm. 1996 225:952-956). These studies suggest that mutant ribosomal-like proteins may be prognostic or diagnostic for cancer and play an important role in regulating the behavior of cancer cells.

In sum, the mechanism(s) by which chromosomal abnormalities associated with rearranging genes are created and the role the enzymes involved in the normal antigen receptor gene (i.e. recombinases) (Croce, C. M. Cell 49:1987 155-169), albeit well-studied, are still poorly understood. Thus, identification of new BPCRs and new recombinases is needed, especially for understanding non-lymphoid type diseases and solid cancer development.

### SUMMARY OF THE INVENTION

An object of the present invention is to provide a Monte Carlo like screening assay for identification of novel transcription factors which comprises the production of random 8 mer DNA sequences and their use in protein binding assays which identify the 8 mer sequence which binds a protein or proteins produced by a selected tissue.

Another object of the present invention is to provide 8 mer consensus sequences identified via the Monte Carlo like screening assays which bind the PCAM-1 protein. These purified nucleic acid sequences are referred to herein as PCAM-1 probe 1 and PCAM-2 probe 2.

Another object of the present invention is to provide isolated, purified nucleic acid sequences encoding PCAM-1 polypeptides or hybridizing with genes encoding PCAM-1 polypeptides.

Another object of the present invention is to provide an amino acid sequence for a PCAM-1 polypeptide.

Another object of the present invention is to provide polyclonal and monoclonal antibodies raised against a PCAM-1 polypeptide which specifically recognize recombinant and native PCAM-1 protein in cells and tissue.

Another object of the present invention is to provide expression vectors and host cells comprising expression vectors which contain a nucleic acid sequence which either encodes a PCAM-1 polypeptide or hybridizes with a gene encoding PCAM-1 polypeptides.

Yet another object of the present invention is to provide methods and kits for diagnosing and/or prognosticating cancer in a patient via detection and/or monitoring of PCAM-1 protein levels in biological samples obtained from the patient. In a preferred embodiment, PCAM-1 is detected in a urine sample via an immunoassay using an antibody raised against the PCAM-1 protein.

### DETAILED DESCRIPTION OF THE INVENTION

Development of markers for the early detection of cancers such as prostate cancer is essential to improved treatment of the cancer. With respect to prostate cancer, it is generally believed that serum prostate specific antigen (PSA) levels are neither sensitive nor specific for identification of patients with prostate cancer (Garnick, M. B. and Fair, W. R. Prostate Cancer. Scientific American, December 1998 75-83). It has been estimated that as many as 25% of men with prostate cancer have normal PSA levels. Thus, development of more sensitive and more specific assays for cancers, including prostate cancer, is clearly needed. Non-invasive and inexpensive urine based screening assays, which would enable implementation through mass community screening programs or in routine clinical examinations, would be particularly useful.

The present invention relates to double-stranded nucleic acid sequences which can be used in screening assays to identify novel DNA binding proteins in nuclear extracts derived from human tissues. The present invention also relates to a novel screening assay, referred to herein as a “Monte Carlo” type screening assay wherein random 8 mer DNA sequences are produced and protein binding assays are employed to identify the 8 mer sequence which binds a protein produced in a selected tissue, i.e. tumor tissue. The Monte Carlo type screening assay was used to identify novel transcription factors over expressed in nuclear protein extracts of tumor tissue. For this assay, 8 mer double stranded DNA probes (n=4096 combinations) were designed. The DNA probes were then used to screen for differences in protein-DNA binding affinity among matched protein extracts from cancer, benign, high grade prostatic intraepithelial neoplasia and seminal vesicle tissue in matched specimens from the same patient (n=11). Binding of proteins was determined via nitrocellulose filters and in electrophoretic mobility gel shift assays (EMSAs). Scintillation counting and phosphoimaging revealed that proteins isolated from nuclear extracts of advanced human prostate cancer tissues specifically bound to two different double stranded nucleic acid sequences comprising PCAM-1 probe 1 (CACGGATG) and PCAM-1 probe 2 (CACAATGA). Proteins extracted from other tissues examined did not bind to these two nucleic acid sequences, indicating they bound a tumor specific protein. One of these sequences, “PCAM-1 probe 2”, was identical to the so called ‘Break Point Cluster Region” or BPCR previously identified in T-cell leukemia cells (Rabbitts, T. H. and Boehm, T. Advances in Immunology 1991 50:119-146). BPCR sequences have previously been associated with chromosomal breakage in T-cells and B-cells and malfunctions in the machinery or proteins associated with these BPCRs may account, in part, for the development of cancerous cells in leukemia and prostate cancer as well. The sequence of “PCAM-1 probe 1” and “PCAM-1 probe 2” are shown below:

“PCAM-1 probe 1⇄:
5′-CACGGATG-3′
3′-GTGCCTAC-5′
“PCAM-1 probe 2”:
5′-CACAATQA-3′
3′-GTGTTACT-5′

The specific DNA sequence identified (i.e. double stranded), CACGGATG and CACAATGA, were employed to screen cDNA libraries developed from PC-3ML prostate cells (Wang et al. Oncology Research 1998 10:219-233). This screening resulted in the identification of phagemid clones which expressed a PCAM-1 ribosomal protein. “PCAM-1” as used herein, refers to the amino acid sequences of purified recombinant or native PCAM-1 protein. Subcloning of the PCAM-1 gene and searched of gene banks showed that this gene exhibits approximately 90 to 95% homology with the human ribosomal protein S2 and the mouse chromosomal protein LLRep3. A nucleic acid sequence expressing this PCAM-1 ribosomal protein comprises SEQ ID NO:1 and a deduced amino acid sequence of this polypeptide comprises SEQ ID NO:2.

Recombinant PCAM-1 protein was demonstrated to bind specifically to “PCAM-1 probe 1” and “PCAM-1 probe 2” in EMSAs (and not to randomly generated 8 mer sequences). DNA protein binding assays were also developed to identify PCAM-1 in biological samples. Using EMSAs, PCAM-1 was detected in tissue extracts from prostate cancer tissue and in urine and serum from human patients. PCAM-1 was not detected in benign or normal prostate tissue or seminal vesicle tissue in these assays. Neither was PCAM-1 detected in the urine or serum of normal individuals without clinical evidence of prostate cancer.

Following development of antibodies, which specifically recognize the PCAM-1 protein, an enzyme linked immuno-sandwich assay or ELISA study was carried out on protein extracts from human tissue and fluids. ELISAs revealed that PCAM-1 was a highly sensitive marker for prostate cancer in studies of crude protein extracts from tissue (Table 1). In these experiments, nuclear protein extracts from microdissected regions of the prostate (n=40 radical prostatectomies examined) expressed significantly elevated levels of PCAM-1 compared to very low levels detected in matching seminal vesicle (SV), benign prostatic hyperplasia (BPH) or high-grade prostatic intraepithelial neoplasm (HGPIN) foci. The amounts of PCAM-1 (μg/mg DNA) appeared to increase as a function of the Gleason Score (GS), indicating the protein may be a stage specific marker for cancer.

TABLE 1
PCAM-1 in microdissected tissues

| Pathology | Tissue PCAM-1 |
|---|---|
| SV (n = 30) | 0 |
| SM (n = 5) | 0 |
| BPH (n = 24) | 0 |
| HGPIN (n = 6) | 0.1 ± 0.03 |
| GS 4 (n = 8) | 1.8 ± 0.31 |
| GS 6 (n = 13) | 10.5 ± 1.15 |
| GS 7 (n = 10) | 20.3 ± 2.06 |
| GS 8-10 (n = 9) | 25.2 ± 3.31 |

*PCAM-1 (μg/mg DNA).

Following radical prostatectomy to remove prostates containing cancerous tissue (n=40 total), the different glandular foci and tissue were dissected from sagittal sections of the prostates. All BPH and HGPIN specimens came from the same prostates exhibiting cancer. Samples were assayed at least 3 times and the data averaged for all the patients in the cohort studied.

Diagnostic tests were conducted to compare urine PCAM-1 levels in patients. Data from these tests are shown in Table 2.

TABLE 2
PCAM-1 Urine Assay (n = 225)

| Diagnosis | PCAM-1 Positive | PCAM-1 Negative |
|---|---|---|
| Prostate Cancer (Biopsy | 24/33 | 9/33 |
| positive GS 4-8) | | |
| Post-Radical | 2/12 | 12/14 |
| Prostatectomy | | |
| BPH | 15/96 | 81/96 |
| Other Prostatic | 1/14 | 13/14 |
| Disorder | | |
| Erectile Dysfunction | 2/13 | 11/13 |
| Volunteer Men | 0/40 | 40/40 |
| (22-53 years old) | | |
| Women | 1/5 | 4/5 |
| | (neurogenic bladder) | |
| Renal Cancer | 1/1 | 0/1 |
| Rectal Cancer | 0/2 | 2/2 |
| Urinary Infection/ | 5/9 | 4/9 |
| Inflammation | | |

Detection limit cut offs were: PCAM-1 positive (>5 ng/ml); PCAM-1 negative (<4 ng/ml). The PCAM-1 levels ranged from 5-93 ng/l in PCAM-1 positive patients and from 0.1-4.0 ng/ml in PCAM-1 negative patients.

As shown in Table 2, the sensitivity of the urine PCAM-1 assay was 73% (i.e. 24/33). In two patients with their prostates removed 3 to 4 years ago (i.e. GS 8-10, stage T3 cancers), the urine PCAM-1 levels were elevated, perhaps due to recurring cancer. These patients are currently under observation to determine whether there is recurring cancer. Conversely, 12 of these patients who were negative for urine PCAM-1 (i.e. GS 5-6, stage T2 cancers). In patients diagnosed with BPH (and no indication of cancer), approximately 16% (n=15/96) exhibited elevated PCAM-1 urine levels. In this cohort of patients, 84% (n=81/96) of the BPH patients were negative for PCAM-1. Of the forty volunteer men, all were negative for PCAM-1. One patient with rectal cancer and 5 patients (n=5/9) with infections or inflammation were positive for PCAM-1, thus indicating that false positives may arise from infections or inflammation.

Thus, these data show that the sensitivity of the PCAM-1 urine assay is 73% for prostate cancer. The overall specificity (i.e. total number of negative patients divided by the total number of patients without disease) was approximately 92% (167/180). Accordingly, it is believed that the PCAM-1 protein is a useful independent diagnostic marker for cancer, and in particular prostate cancer.

In one aspect of the present invention, nucleic acid sequences which encode the PCAM-1 protein and the deduced amino acid sequence of the PCAM-1 protein encoded by these nucleic acid sequences are provided. An exemplary nucleic acid sequence encoding the PCAM-1 protein and an exemplary deduced amino acid sequence are depicted in SEQ ID NO:1 and SEQ ID NO:2, respectively. “Nucleic acid sequence” as used herein refers to an oligonucleotide, nucleotide or polynucleotide and fragments or portions thereof and to DNA or RNA of genomic or synthetic origin which may be single- or double-stranded and represent the sense or antisense strand. The terms “amino acid sequence”, “polypeptide” or “protein” as used herein refers to an amino acid sequence of a naturally occurring protein molecule which has been isolated and purified and is associated with the PCAM-1 protein.

The present invention also relates to expression vectors and host cells containing expression vectors which comprise these nucleic acid sequences. Expression vectors and host cells, which can be transfected with an expression vector, are well known in the art. Methods for incorporating a selected nucleic acid sequence such as that of the present invention into a vector and ultimately to a host cells are also well known.

The nucleic acid and amino acid sequences of the present invention are useful in developing screening assays for detection of PCAM-1 protein in biological samples. As demonstrated herein, in one embodiment, antibodies can be raised against the PCAM-1 protein and used in an immunoassay such as an ELISA to detect PCAM-1 protein in a biological sample such as tissue, sputum, urine or serum. Monoclonal or polyclonal antibodies can be raised against this protein in accordance with well known procedures. Alternatively, labeled nucleic acid probes can be prepared from the nucleic acid sequences of the present invention and used in EMSAs to detect PCAM-1 in nuclear extracts of tissue biopsy samples.

Thus, another aspect of the present invention relates to methods and kits for detection of PCAM-1 in biological samples. As demonstrated herein, detection of PCAM-1 levels in a biological sample of a patient is useful in diagnosing and prognosticating cancers, and in particular prostate cancer, in the patient. In the method of the present invention a biological sample is obtained from a patient and then contacted with a means for detecting PCAM-1 in the biological sample. In one embodiment, this means can comprise an antibody raised against the PCAM-1 protein, which is capable of detecting PCAM-1 protein in biological samples such as tissues, sputum, serum and urine. In another embodiment, this means can comprise a labeled nucleic acid probe such as CACGGATG which is capable of detecting PCAM-1 protein in biological samples such as tissue biopsies. Accordingly in the kits of the present invention a means for detecting PCAM-1 protein in a sample and a PCAM-1 protein standard is provided. Means for detecting PCAM-1 protein may comprise an antibody raised against the PCAM-1 protein or a labeled nucleic acid probe capable of binding to the protein. The presence of PCAM-1 in the biological sample is indicative of the patient having cancer, and in particular prostate cancer. Methods and kits of the present invention can also be used in patients with prostate cancer to assess their prognosis and evaluate treatments by monitoring changes in levels of PCAM-1 in the patient over time. Increases in the level of PCAM-1 over time is indicative of the cancer progressing while decreases in the level of PCAM-1 over time is indicative of regression of the cancer.

Further, it is believed that these methods and kits for detecting PCAM-1 protein levels may also be useful in diagnosing and prognosticating other types of cancer, inflammatory conditions, infections and genetic mutations.

What is claimed is:

1. A Monte Carlo like screening assay for identification of proteins in a selected tissue comprising producing random 8 mer double stranded DNA sequences and employing protein binding assays to identify the 8 mer sequence which binds a protein produced in the selected tissue.

2. An isolated, purified nucleic acid sequence which binds PCAM-1 protein.

3. The isolated, purified nucleic acid sequence of claim 2 comprising the sequence CACGGATG.

4. The isolated, purified nucleic acid sequence of claim 2 comprising the sequence CACAATGA.

5. An isolated, purified nucleic acid sequence which encodes a PCAM-1 polypeptide or hybridizes with a gene encoding a PCAM-1 polypeptide.

6. The isolated, purified nucleic acid sequence of claim 5 comprising SEQ ID NO:1.

7. The isolated, purified nucleic acid sequence of claim 5 wherein the encoded PCAM-1 polypeptide comprises SEQ ID NO:2.

8. An isolated, purified PCAM-1 polypeptide.

9. The isolated, purified PCAM-1 polypeptide of claim 8 comprising SEQ ID NO:2.

10. An antibody raised against the PCAM-1 polypeptide of claim 8.

11. An expression vector comprising a nucleic acid sequence which encodes a PCAM-1 polypeptide or hybridizes with a gene encoding a PCAM-1 polypeptide.

12. A host cell comprising the expression vector of claim 11.

13. A method for diagnosing and prognosticating cancer in a patient comprising obtaining a biological sample from a patient and detecting or monitoring PCAM-1 protein levels in the biological sample wherein the presence of PCAM-1 protein in the biological sample is indicative of the presence of or progression of cancer in the patient.

14. The method of claim 13 wherein the cancer is prostate cancer.

15. A kit for diagnosing and prognosticating cancer in a patient comprising:
(a) a PCAM-1 standard; and
(b) a means for detecting PCAM-1 protein in biological samples.

16. The kit of claim 15 wherein the cancer is prostate cancer.


SEQUENCE LISTING (2 sequences)

SEQ ID NO: 1 - DNA, 911 bp, Homo sapiens
gcacgagggatgacgccggtgcagcgggggggcccgggggccctggtggccctgggatggggaaccgcggtggcttccgcggaggtttcggcagtggcatccggggccggggtcgcggccgtggacggggccggggccgaggccgcggagctcgcggaggcaaggccgaggataaggagtggatgcccgtcaccaagttgggccgcttggtcaaggacatgaagatcaagtccctggaggagatcactctcttctccctgcccattaaggaatcagagatcattgatttcttcctgggggcctctctcaaggatgaggttttgaagattatgccagtgcagaagcagacccgtgccggccagcgcaccaggttcaaggcatttgttgctatcggggactaccatccgtggggccatcatcctggccaagctctccatcgtccccgtgcgcagaggctactgggggaacaacatcggcaaggcccacactgtccgttgcaaggtgacaggccgctgcggctctgtgctggtacgcctcatccctgcacccaggggcactggcatcgtctccgcacctgtgcctaagaagctgctcatgatggctggtatcgcaatggccacgtcggtctgggtgttaagtgctccaaggaggtggccaccgatgactgctacacctcagcccggggctgcactgccaccctggcaacttcgccaaggccacctttgatgccatttctaagacctacagtcacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaagaaaaataaa

SEQ ID NO: 2 - protein, 276 aa, Homo sapiens
MGNRGGFRGGFGSGIRGRGRGRGRGRGRGRGAGRGGKAEDKEWMPVTKLGRLVKDMKIKSLEEITLFSLPIKESEIIDFFLGASLKDEVLKIMPVQKQTRAGQRTRFKAFVAIGDYVGLGVKCSKEVATAIRGAIILAKLSIVPVRRGYWGNNIGKAHTVRCKVTGRCGSVLVRLIPAPRGTGGIVSAPKKLLMMANGHAGIDDCYTSARGCTATLGNFAKATFDAISKTYSYLTPDLWKETVFTKSPYQEFTDHLVKTHTRVSVQRTQAPAVATT